(19) United States
(12) Patent Application Publication
(10) Pub. No.: US 2023/0226082 A1
(43) Pub. Date: Jul. 20, 2023

(54) METHODS AND FORMULATIONS FOR TREATING AND PREVENTING LONG HAUL CORONAVIRUS (LONG COVID) SYMPTOMS AND SEQUELAE

(71) Applicant: Bioved Pharmaceuticals, Inc., San Jose, CA (US)

(72) Inventors: Deepa Chitre, LOS GATOS, CA (US); Debendranath Dey, FREMONT, CA (US); Satej R Nadkarni, San Jose, CA (US)

(73) Assignee: Bioved Pharmaceuticals, Inc., San Jose, CA (US)

(21) Appl. No.: 17/985,097

(22) Filed: Nov. 10, 2022

Related U.S. Application Data
(63) Continuation-in-part of application No. 17/306,937, filed on May 3, 2021, now Pat. No. 11,628,197.
(60) Provisional application No. 63/019,412, filed on May 3, 2020.

(51) Int. Cl. A61K 31/585; A61K 31/19; A61K 31/12; A61K 36/9068; A61K 36/9066; A61K 36/324; A61K 36/81; A61K 31/675; A61K 31/4025; A61K 31/375; A61K 33/30; A61K 31/198; A61K 9/20; A61K 9/16; A61P 31/14; A61P 27/00

(52) U.S. Cl. CPC A61K 31/585; A61K 31/19; A61K 31/12; A61K 36/9068; A61K 36/9066; A61K 36/324; A61K 36/81; A61K 31/675; A61K 31/4025; A61K 31/375; A61K 33/30; A61K 31/198; A61K 9/2059; A61K 9/2009; A61K 9/2027; A61K 9/1611; A61K 9/1652; A61P 31/14; A61P 27/00; A61K 2236/333; A61K 2236/53

(57) ABSTRACT

Methods and formulations for treating and preventing LONG COVID sequelae are provided. An example method treats loss of smell and taste from SARS-CoV-2 infection, including assessing the patient, administering a composition with dosage between 1750-3600 mg per day, wherein the composition comprises a mixture of at least a withanolide-A, a boswellic acid, a [6]-gingerol, and a curcuminoid. Another example method treats memory loss or brain fog from SARS-CoV-2 infection, including assessing the patient, and administering a regenerative and neuroprotective composition comprising a mixture of withanoside VI, withanolide A, a Boswellia serrata plant, a Zingiber officinale rhizome, and a Curcuma longa rhizome. The compositions were found effective, achieving statistical significance in a multi-center, randomized, double-blind, placebo-controlled phase III clinical trial.

### RELATED APPLICATIONS

This continuation-in-part patent application claims the benefit of priority to U.S. patent application Ser. No. 17/306,937 to Chitre et al., filed May 3, 2021 and incorporated by reference herein in its entirety, which in turn claims priority to US Provisional Patent Application No. 63/019,412 to Chitre et al., filed May 3, 2020 and incorporated by reference herein in its entirety.

### BACKGROUND

Aggressive global vaccination efforts, widespread public healthcare measures, and a better understanding of treatment modalities provide a significant improvement in the management of the SARS-CoV-2 pandemic. However, in many developing countries and also among certain populations in developed nations, the disease and its variants continue to cause significant morbidity, mortality, and economic impact.

Long COVID or long-haul COVID has arisen as a set of long-term symptoms and health conditions that persist or increase after an initial, acute bout of COVID-19 disease. Long COVID has also been called post-COVID-19 syndrome, post-COVID-19 condition, post-acute sequelae of COVID-19 (PASC), and chronic COVID syndrome (CCS). Long COVID is thus a condition characterized by long-term health problems persisting or appearing after the typical recovery period for COVID-19 disease. Long COVID may affect nearly every organ system, causing further conditions (sequelae) including respiratory system disorders, nervous system disorders, neurocognitive disorders, mental health disorders, metabolic disorders, cardiovascular disorders, gastrointestinal disorders, musculoskeletal pain, and anemia, for example.

The most commonly reported symptoms of long COVID are fatigue and memory problems. Many other symptoms have also been reported, including malaise, headaches, shortness of breath, anosmia (loss of smell), parosmia (distorted smell), muscle weakness, low fever, and cognitive dysfunction.

Evidence also suggests that infection with COVID-19 disease may predispose individuals to both venous and arterial thromboembolism due to excessive inflammation, hypoxia, immobilization, and diffuse intravascular coagulation. This is estimated to happen in up to 31% of patients, hence it is important to prevent these by giving adequate preventive and prophylactic treatment.

Autopsy analyses of patients with COVID-19 disease complicated by Acute Respiratory Distress Syndrome (ARDS) show highly activated cytotoxic T-cells, resulting from hyperactivation of the immune system. A significant surge of Interleukin-6 (IL-6), Tumor Necrosis Factor-alpha (TNF-α), and other cytokines are thought to be the mediators of this enhanced T-cell activity.

### BRIEF DESCRIPTION OF THE DRAWINGS

FIG. 1 is a flow diagram of the layout of a Phase III clinical study conducted to assess the efficacy and safety of an example formulation for treating LONG COVID-19 sequelae caused by SARS-CoV-2 infection.

FIG. 2 is a table showing subject disposition for the Phase III clinical study.

FIG. 3 is a table showing demographic characteristics for the Phase III clinical study.

FIG. 4 is a table showing a primary efficacy analysis, for the intention-to-treat infected (ITTI) population of the Phase III clinical study.

FIG. 5 is a table showing a primary efficacy analysis, for the per protocol (PP) population of the clinical study.

FIG. 6 is a table showing demographic characteristics such as gender, mean age, race, body weight, height, and BMI of groups of patients in the Phase III clinical study.

FIG. 7 is a table showing that a mean difference of reduction in the duration of illness between a treatment group receiving an example formulation described herein versus a placebo group was statistically significant at p=0.036.

FIG. 8 is a table showing a primary and secondary efficacy analysis with severity scores of symptoms.

FIG. 9 is a chart showing mean Quality-of-Life scores over a study period of 14 days.

FIG. 10 is a table showing outcomes and primary & secondary efficacy analysis of 53 patients given a combination of the example formulation and remdesivir versus 47 patients given a combination of placebos and remdesivir.

FIG. 11 is a table showing alleviation of symptoms in 53 patients given a combination of the example formulation and remdesivir versus 47 patients given a combination of placebos and remdesivir.

FIG. 12 is a diagram showing example major and minor high-performance liquid chromatography (HPLC) peaks of select metabolites of Withania somnifera in a custom example formulation.

FIG. 13 is a flow diagram showing an example process for making a batch size of approximately 220,000 tablets of example formulations.

FIG. 14 is a diagram showing reduction in erythrocyte sedimentation rate (ESR) during COVID-19 disease using the example formulation for 16 weeks, and achieving statistical significance after 32 weeks of cumulative therapy.

FIG. 15 is a diagram showing reduction in C-Reactive Protein (CRP) during COVID-19 disease using the example formulation for 16 weeks, and achieving statistical significance after 32 weeks of cumulative therapy.

FIG. 16 is a flow diagram showing an example method for treating at least a partial loss of smell or taste secondary to a SARS-CoV-2 infection.

FIG. 17 is a flow diagram showing an example method for treating a memory loss or a brain fog symptom secondary to a SARS-CoV-2 infection.

### DETAILED DESCRIPTION

### Overview

This application describes methods and formulations for treating long haul coronavirus symptoms and sequelae. The example methods and formulations can also be used prophylactically, to help prevent vulnerability to COVID-19 disease and exacerbation of its symptoms.

A Phase III randomized, double-blind, placebo-controlled clinical study in 176 subjects with moderate SARS-CoV-2 infection determined that example formulations, described herein, can be administered orally to shorten the duration and decrease the individual impacts of various long COVID symptoms and sequelae.

### Phase III Clinical Study

A multi-center, randomized, double-blind, placebo-controlled Phase III clinical trial was conducted from September 2020 to April 2021 to assess the efficacy and safety of an example formulation used to treat COVID-19 disease and its symptoms as caused by SARS-CoV-2 infection. The clinical study assessed the example formulation, combined with current Standards of Care, and also assessed the impact of the example formulation on biomarkers in subjects with uncomplicated moderate SARS-CoV-2 infections causing COVID-19 disease. The double-blind placebo-controlled clinical trial described herein (hereinafter, “clinical study”) used a combination of modified derivatives Ayurvedic herbs and plant parts (“example formulation”) for treatment of hospitalized COVID-19 subjects.

In an implementation, the example formulation contains specialized or modified extracts of four medicinal plants: Ashwagandha (Withania somnifera, family Solanaceae), Shallaki (Boswellia serrata, family Burseraceae), Ginger (Zingiber officinale, family Zingiberaceae) and Turmeric (Curcuma longa, family Zingiberaceae).

Results of the clinical study show that the example formulation is therapeutic for treating multiple physiological sequelae associated with long COVID. The plant materials used in the clinical study have been identified, authenticated, and deposited in a Government of India Herbarium, namely Central Council for Research in Ayurvedic Sciences (CCRAS) based in Pune, India. The voucher numbers are W. somnifera (4390), B. serrata (4391), C. longa (4392) and Z. officinale (4393). The use of resources and work was been approved by the National Biodiversity Authority of India.

In the double-blind placebo-controlled clinical study, the example formulation provided improvement in both the clinical outcomes of patients with COVID-19 disease, and in symptoms associated with long COVID. In an implementation, the example formulation can reduce the duration of illness, severity of various symptoms, boost immunity, and prevent the incidence of COVID-19 complications.

In the clinical study, hospitalized patients were randomly assigned to receive either the example formulation or placebo tablets for 14 days, at four sites in India during late 2020 to early 2021. Among 208 randomized subjects, 175 completed the study. In a group receiving the example formulation, the mean reduction in duration of illness (p=0.036), and severity scores showing alleviation of several symptoms such as fever, cough, and smell and taste disorders, were statistically significant (p<0.05).

A subset analysis of subjects treated with or without remdesivir, an example antiviral medication used as a standard of care in the clinical study, showed mean reduction in duration of illness in a group receiving the example formulation with remdesivir (p=0.030) and reduction in their severity scores (p<0.05).

The mean difference criterion with respect to levels of the immune protein Interleukin-6, as a biomarker of inflammation, was statistically significant (p=0.042) in patients using the example formulation without remdesivir. Thus, in an implementation, the example formulation may reduce duration of illness, symptoms severity, Interleukin-6 levels, and prevent the incidence of COVID-19 complications in moderate COVID-19 cases. The formulation may also have an adjunctive effect with other standards of care. (Clinical Trials Registry of India: CTRI/2020/09/027817).

The four cultivated plants in the example formulation may be extracted from the respective plants in a manner that can modify the usual ratios of bioactive species available from each plant. Each extract may be standardized to a desired composition of desired bioactive species using High-Performance Liquid Chromatography (HPLC), High-Performance Thin-Layer Chromatography (HPTLC), and Spectrophotometry to assay the results and achieve the exact percentages of desired bioactive compounds.

Each extract was also tested for absence of pesticides, residual solvent levels, and heavy metals.

### Clinical Study Design

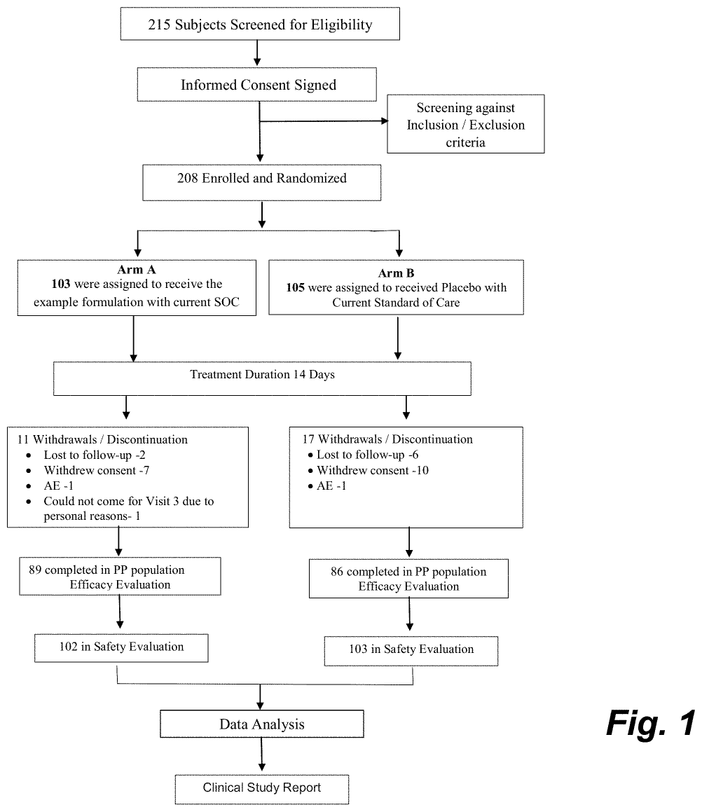

FIG. 1 shows a layout of the clinical study introduced above, a multi-center, randomized, double-blind, placebo-controlled Phase III clinical trial conducted to assess the efficacy and safety of an example formulation used to treat COVID-19 disease and its sequelae caused by SARS-CoV-2 infection. The clinical study assessed the example formulation when used with and without current standards of care (SOC), and also studied the impact of the example formulation on inflammatory biomarkers in subjects with uncomplicated, moderate SARS-CoV-2 infections. Since the clinical study was conducted from September 2020 to April 2021 during the first and second waves of the COVID-19 pandemic in India, the second wave largely consisted of the Delta or B.1.617 variant of the SARS-CoV-2 virus. The B.1.617 variant was highly transmissible, and led to more than 400,000 new reported cases per day and a record number of deaths.

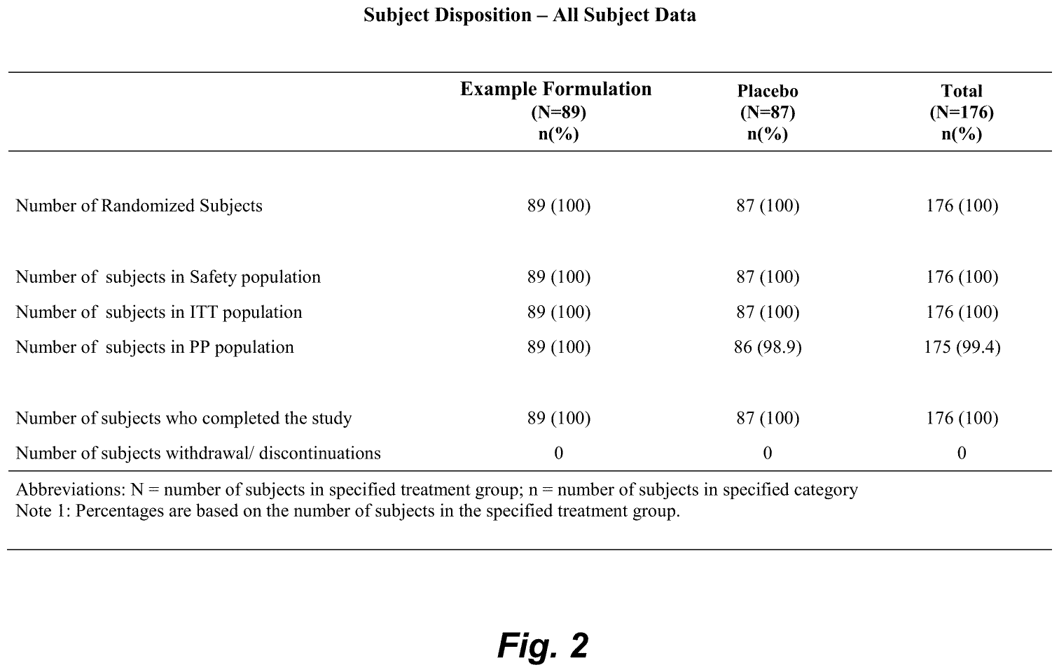

FIG. 2 shows subject disposition for the Phase III clinical study. As above, the majority of patients in the clinical study were recruited during the second wave of the COVID pandemic in India. A further subset analysis was conducted on subjects who were treated with remdesivir as a SOC, using the same primary and secondary end points as patients treated without remdesivir as an example antiviral.

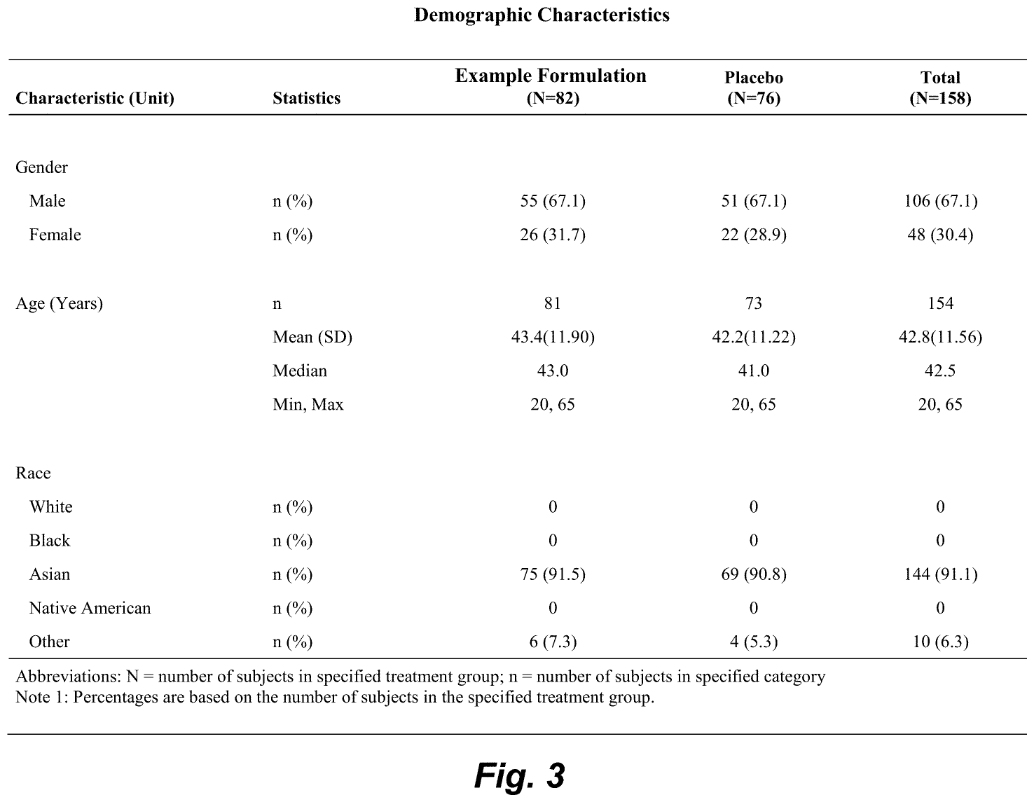

FIG. 3 shows demographic characteristics for the Phase III clinical study. Sample size was based on comparative trials in the general population near the same time as the clinical study. With a viable sample of subjects, a one-sided log rank test with an overall sample size of 170 subjects (85 in the treatment group and 85 in the placebo group) would be expected to achieve 80% power at a 0.025 significance level to detect a hazard ratio of 1.67. Considering 6% dropouts in the study, a total 180 subjects (90 in the treatment group and 90 in the placebo group) were enrolled in the clinical study.

The clinical study was conducted in compliance with the principles of the Declaration of Helsinki, International Council for Harmonization—Good Clinical Practice guidelines, and regulatory guidelines of the Indian Council of Medical Research (ICMR) and the Department of AYUSH. All required study documentation was archived as required by regulatory authorities. The study was managed by a global Clinical Research Organization (CRO) based in San Jose, California. The protocol was also filed with the Drug Control General of India, ICMR and AYUSH. The study is registered in Clinical Trials Registry of India with details as CTRI/2020/09/027817. Approval of the protocol, protocol amendments, and informed consent forms by the Institutional Review Boards, Independent Ethics Committees (EC) were mandatory. Subject participation was voluntary.

The clinical study was conducted at four tertiary care in-patient hospital study centers in a competitive enrollment method. Male and female subjects, aged 18-65 years with moderate COVID-19 infections who were already hospitalized, with temperature 38° C. (100.4° F.), plus at least one respiratory symptom (nasal congestion, sore throat, cough, or breathing difficulty), and at least one constitutional symptom (aches/pains, fatigue, headache, chills, or sweats) were screened. Only those subjects with confirmed SARS-CoV-2 infection by Reverse Transcription—Polymerase Chain Reaction (RT-PCR) prior to a first visit were included. All female subjects of child-bearing potential and male subjects and their spouse/partner had to agree to use a medically acceptable method of contraception throughout the entire study period, and for 30 days for females, and 90 days for males, after study discontinuation.

Subjects with severe COVID-19 infection requiring intensive inpatient treatment were excluded from this clinical trial. Subjects requiring systemic antiviral therapy prior to screening or those on immunomodulators, interferon inducers, homeopathic, or hormonal therapy (other than hormone replacement therapy) were also excluded. Use of corticosteroids as part of the SOC was permitted. Subjects were excluded who had: uncontrolled hypertension (systolic blood pressure>140 mm Hg or diastolic blood pressure>90 mm Hg), diabetes, asthma (any current or recent, not childhood if resolved), COPD, cardiac disorders, hepatic disorders, renal disorders (e.g., eGFR<60) and hematopoietic disorders, neurological disease, compromised immune system (including patients on immunosuppressant therapy, or those with cancer within the past 5 years or human immunodeficiency virus [HIV] infection), endocrine disorders (including thyroid disorders), or anatomical nasal obstruction (including polyps and septal deviation). Other exclusion criteria were clinically obese subjects with BMI 40, recent history (within 6 months) of alcoholism or substance abuse, participation in another clinical trial within 1 month or during this clinical study, pregnant or breast-feeding female subjects, known allergy to components of the example formulation, previous history of difficulty swallowing capsules or tablets, or any other disease or condition which, in the opinion of the investigator, restricted or impeded participation in the clinical study or affected the clinical results for extraneous reasons.

### Efficacy Endpoints

The primary endpoint was improvement in reducing the duration and severity of COVID-19 disease and its symptoms compared to placebos. The example formulation, or placebos, were started within 48 hours and no more than 96 hours after onset of COVID19 symptoms, per individual. A standard severity score was used on a 0-3 scale to determine an overall score. Subjects participating in the trials were required to self-assess the COVID-19 associated symptoms as “none or 0,” “mild or 1,” “moderate or 2,” and “severe or 3” using Symptom Assessment Cards (SAC), a symptom assessment tool employed in the clinical study for applying uniform self-assessment of symptoms. The duration of illness and time to improvement were calculated from the time of initiation of treatment to the time when all symptoms including fever, nasal congestion, sore throat, cough, breathing difficulty, aches and pains, fatigue, headaches, chills/sweat, diarrhea, vomiting, smell and taste disorders were assessed as “none” or “mild” (score 1 or 0), and stable for at least 24 hours. Alleviation of fever was defined as an oral temperature lower than 37.3° C. (99.1 ° F.) and stable for at least 24 hours.

Secondary endpoints were the severity scores, and reduction in the duration measuring alleviation of individual symptoms from time 0 to the time at which the symptom was less than or equal to mild and stable for at least 24 hours. The percentage of subjects experiencing alleviation of COVID-19 symptoms at every 24 hours post first dose to the end of day 14, and the average of the severity scores every 24 hours post first dose to the end of day 14 were studied. A questionnaire for self-assessment of Quality-of-Life (QoL) was used to gauge patient well-being and subjective assessment of status. The hospitalization rate of subjects with severe COVID-19 symptoms requiring intensive inpatient treatment, and the percentage of subjects that experienced complications was notated. Both the Symptom Assessment Cards and the Quality-of-Life cards were filled out by the patients, verified by the respective Study Coordinator at each site, and the data were entered into the electronic Case Report Forms (e-CRF). These data were further reviewed by the Clinical Research Associates at each site from the Clinical Research Organization (CRO), and the source data were verified for accuracy and proper entry into the e-CRFs.

Exploratory endpoints were: improvement in the biomarkers IL-6, TNF, CRP, Erythrocyte Sedimentation Rate (ESR), and Lactate Dehydrogenase (LDH). Safety was assessed by monitoring and recording all adverse events (AEs), regular physical examinations, hematology results, lab chemistry, and 12-lead-electrocardiograms. Any other medications for COVID-19 other than those allowed under the adopted SOC were not permitted.

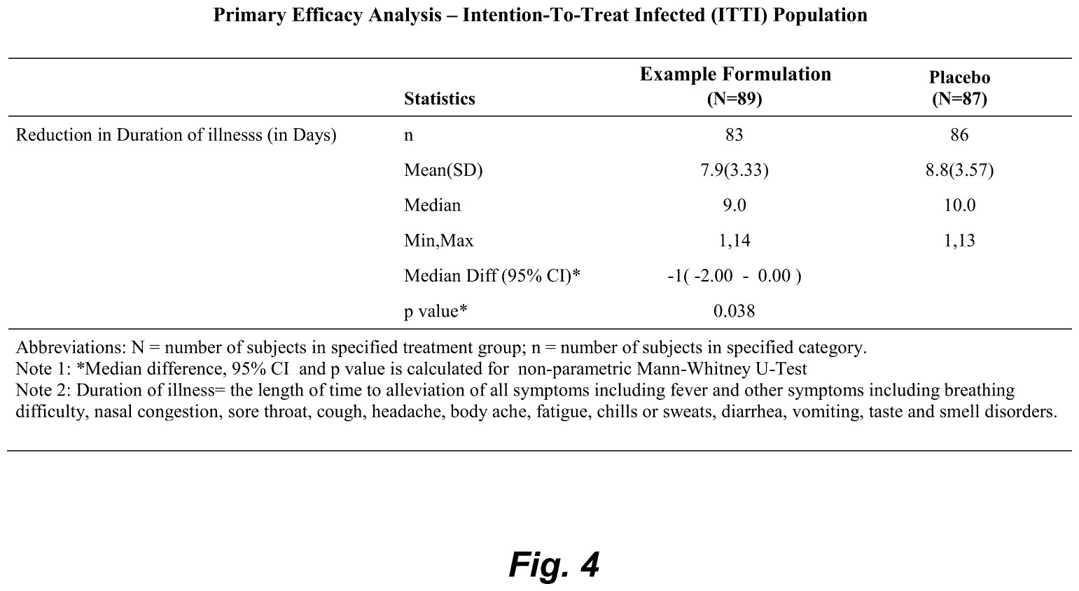

FIG. 4 shows a primary efficacy analysis, for the intention-to-treat infected (ITTI) population of the clinical study.

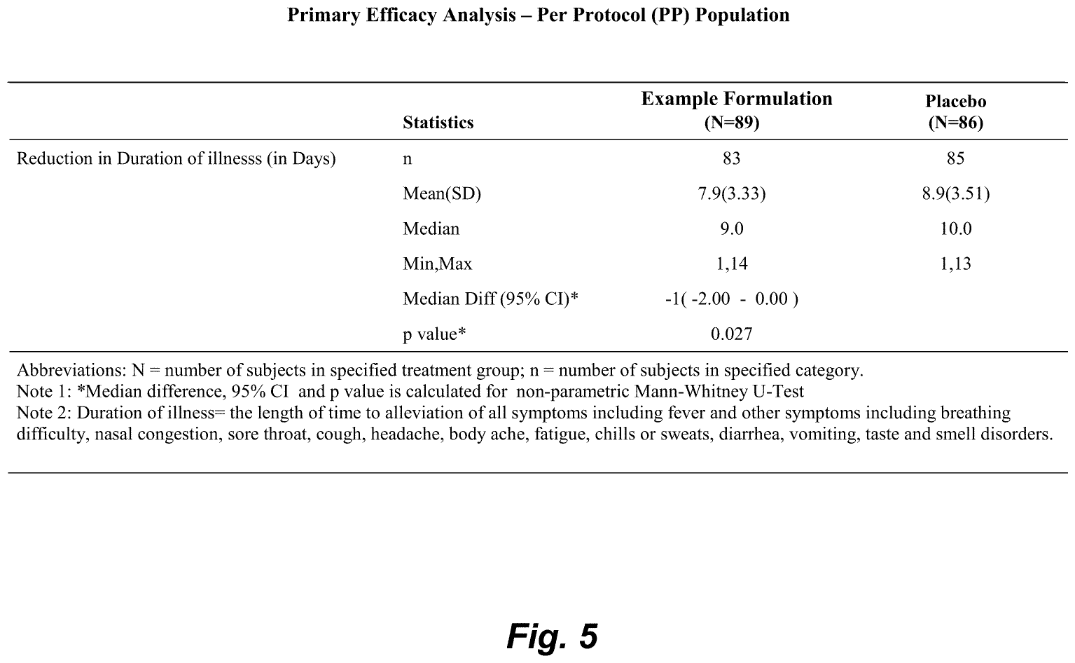

FIG. 5 shows a primary efficacy analysis, for the per protocol (PP) population of the clinical study.

### Statistical Analysis

All statistical analyses were performed using SAS® Version 9.4 or higher (SAS Institute Inc., Cary, N.C.). The mean duration of illness and duration of alleviation of individual symptoms were compared using the Mann-Whitney U test between the treatment group using the example formulation and the placebo group. For the change from baseline summaries, the baseline value was the value or measurement recorded at the baseline visit. Mean reduction in duration, with respect to alleviation of individual symptoms, for example including fever, from time 0 (first administration of the “study medication”) to the time at which the given symptom was less than or equal to mild, and stable for at least 24 hours, was compared between the treatment group and the placebo group using the nonparametric Mann-Whitney U test. The proportion of subjects experiencing alleviation of COVID-19 symptoms at every 24 hour interval, post first dose to the end of day 14, was compared between the treatment group and the placebo group using a chi-squared test. The average of severity scores at every 24 hour interval post first dose to the end of day 14 was compared between the treatment group and the placebo group using repeated measures ANOVA.

Scores of the Quality-of-Life assessments based on the self-assessment questionnaires were compared between the treatment group and the placebo group using repeated measures ANOVA. The proportion of subjects experiencing complications or worsening of symptoms was compared between the treatment group and the placebo group using the chi-squared test. A summary of Adverse Events (AEs) was analyzed, including the number and percentage of subjects with any adverse events, treatment emergent adverse events (TEAEs), serious adverse events (SAEs), drug-related AEs, drug related SAEs, discontinuations and their incidence.

A p-value<0.05 was considered to be statistically significant. All analyses were performed according to ITT (Intent-to-Treat), PP (Per Protocol), and Safety Analysis populations. The PP analyses were reported for all efficacy endpoints. The Adverse Events results were based on a Safety set analysis.

### Clinical Study Results

A total of 215 subjects were screened, 208 were randomized, 3 subjects withdrew consent, and 205 subjects took 1776 mg of the example formulation, or else a placebo, twice per day after meals for 14 days. 103 subjects received the example formulation, and 102 subjects received placebos. 196 subjects were analyzed under an Intent-to-Treat (ITT) set, 175 subjects were analyzed under a Per Protocol (PP) set, and 205 subjects were analyzed under a Safety set. 175 subjects completed the study of which 89 subjects received the example formulation within the context of the SOC, and 86 subjects received placebos with the SOC. A total of 30 subjects did not complete the 14 days of treatment and were not included in final PP analysis. Of these, 2 subjects withdrew consent after randomization, 2 subjects withdrew due to adverse events, 1 subject was unable to come for Visit 3 due to personal reasons, 17 subjects withdrew consent at various times during the study, and 8 subjects were lost to follow up.

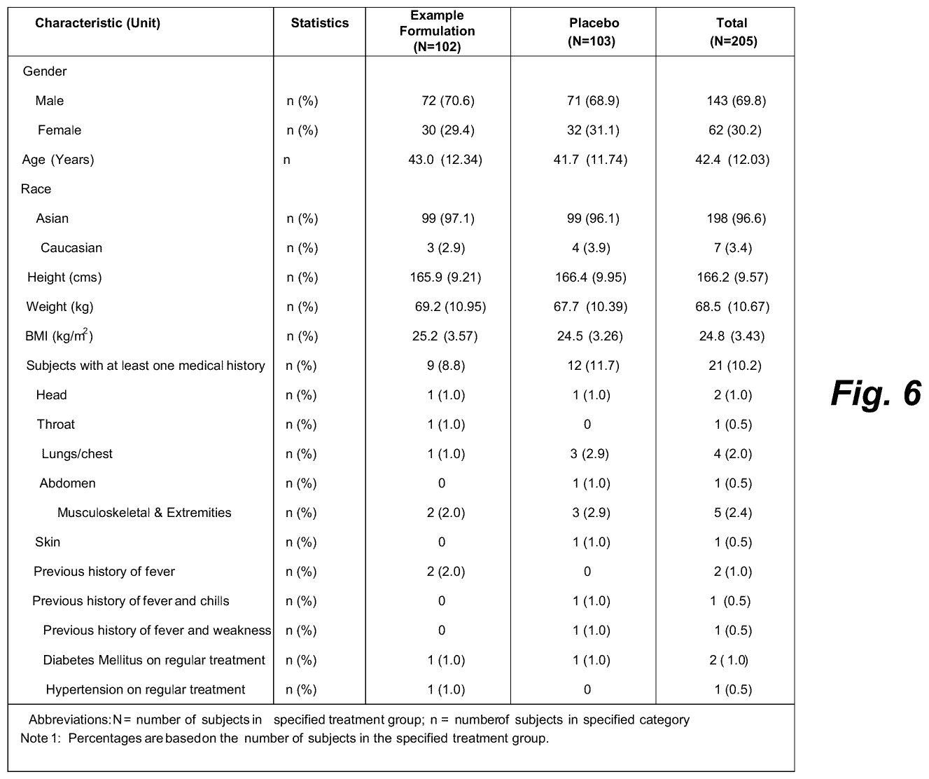

FIG. 6 shows that all demographic characteristics such as gender, mean age, race, body weight, height, and BMI were well balanced between the groups. The Standard of Care (SOC) treatment was according to the investigators' discretion following the applicable Indian Council of Medical Research (ICMR) guidelines at that time, and consisted mainly of analgesics, antibiotics, cough syrups and corticosteroids. Since the hospitalized subjects had moderate Covid-19 disease, almost all received steroids. The average dose of steroids was 4 mg once or twice per day. More than 50% subjects took remdesivir as part of the Standard of Care.

### Primary and Secondary Efficacy Endpoints Results

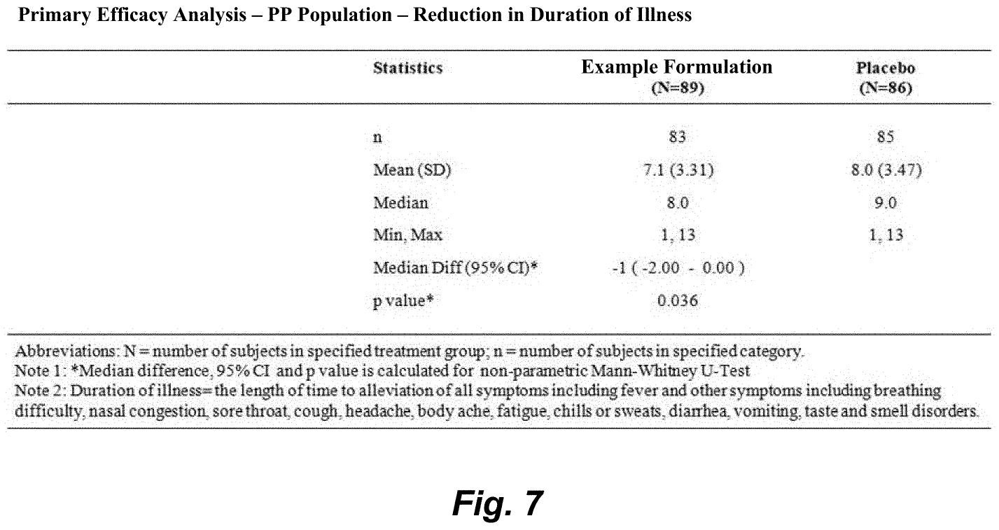

FIG. 7 shows that the mean difference of reduction in the duration of illness between the treatment group and the placebo group was statistically significant at p=0.036.

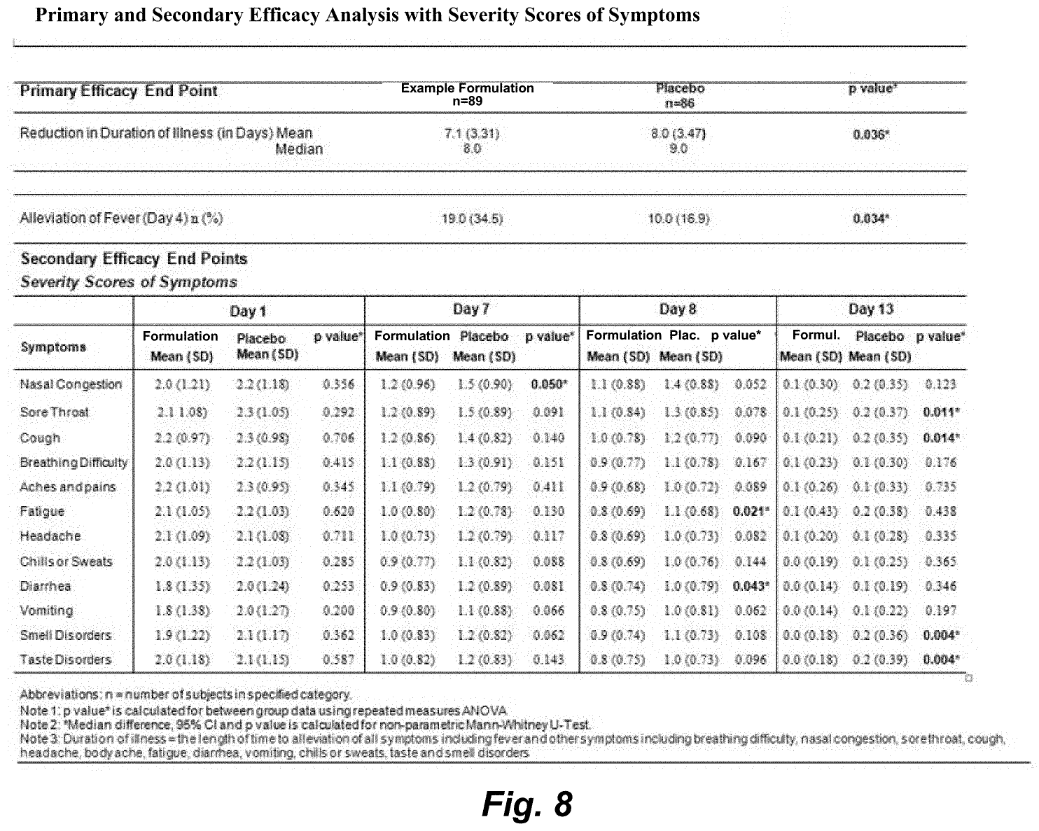

FIG. 8 shows patients experiencing significant alleviation of fever on day 4, at p=0.034. The mean Severity Scores of Symptoms in FIG. 8, including nasal congestion, sore throat, cough, fatigue, headache, diarrhea, and smell and taste disorders, had a greater decrease in the treatment group than in the placebo group, and symptom reductions were statistically significant. In several of the parameters, on several days, the patients showed significant improvement, but did not achieve statistical significance.

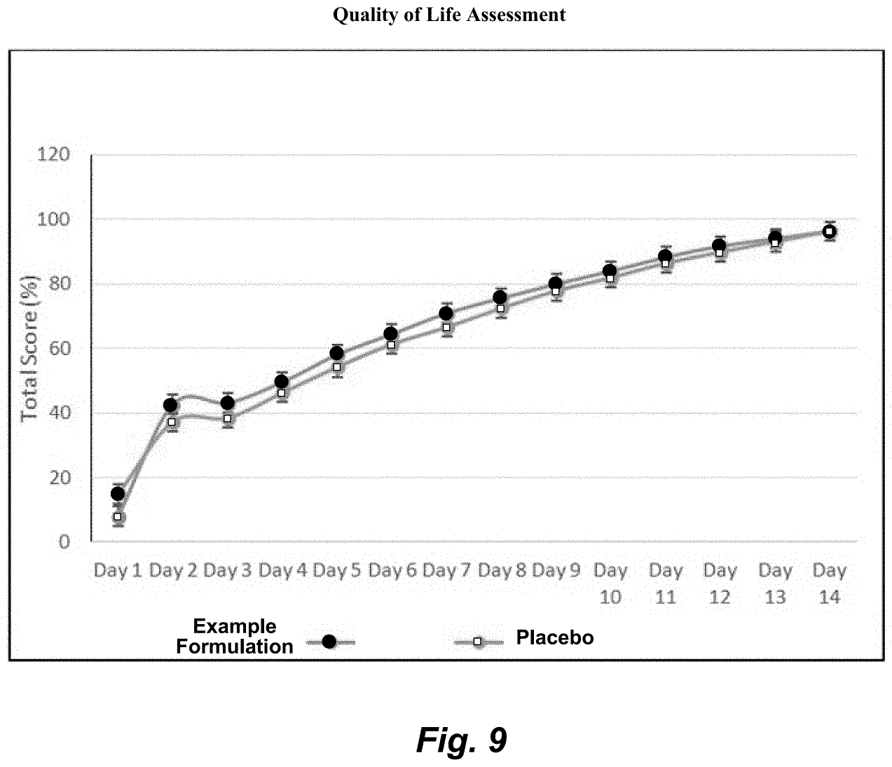

FIG. 9 shows that the mean Quality-of-Life scores improved gradually in both groups by day 14, but did not reach the threshold set for statistical significance. Some biomarkers as described above also showed improvement but did not reach statistical significance. There were no complications or significant protocol deviations during the entire conduct of the study.

### Safety and Adverse Events

A total of five (2.4%) of the hospitalized subjects reported at least one adverse event (AE). A total of 13 adverse events were reported by those 5 subjects. Most of the adverse events were of moderate severity. All the reported adverse events were unrelated to the study medication, the example formulation. One patient each from the treatment group and the placebo group withdrew permanently. Out of the 13 adverse events, seven adverse events were resolved, one adverse event was ongoing at the time of final reporting, and the status of five adverse events was unknown. No serious adverse events and no deaths were reported in the clinical study. Most other reported adverse events were inflammation in limbs, increase in glucose level, increase in IL-6, hypertension, and vomiting (0.5%). While the biomarkers were exploratory endpoints in the clinical study, extremely high values were considered as adverse events by the principal investigators and recorded as such. Hematology, biochemistry, urinalysis, and 12-lead-electrocardiograms did not show any noteworthy changes from normal.

### Efficacy Subset Analysis with Remdesivir

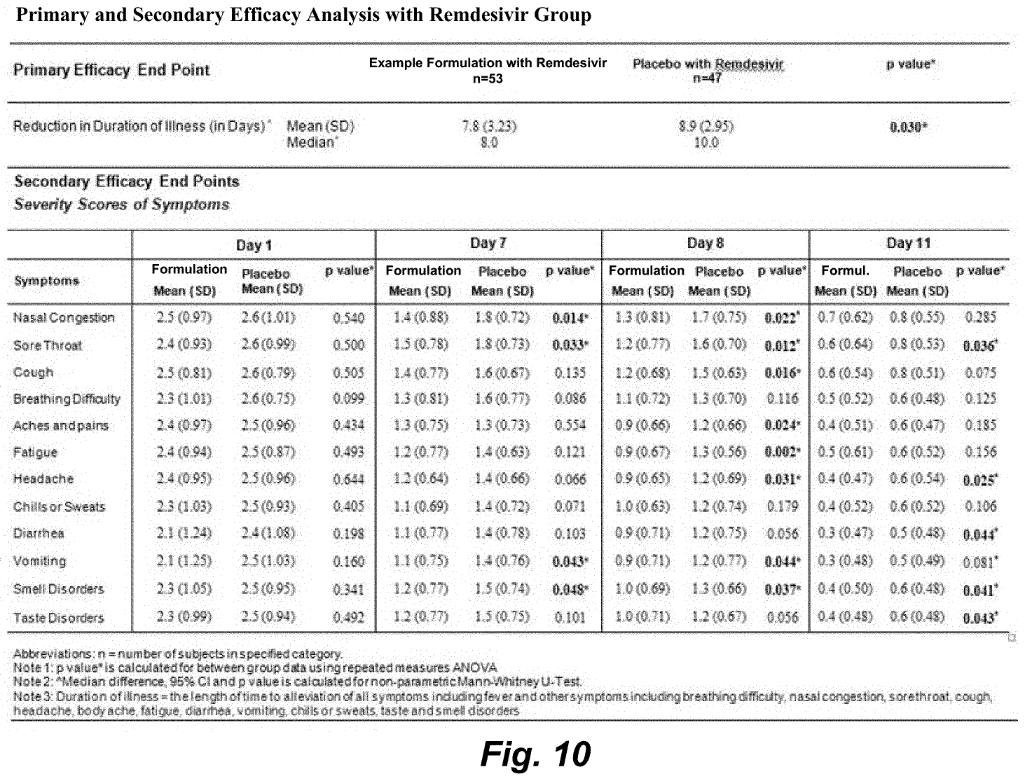

FIG. 10 shows outcomes, and primary and secondary efficacy analysis between 53 patients given a combination of the example formulation and remdesivir, an antiviral, and 47 patients given a combination of placebos and remdesivir. In the patients receiving the example formulation plus remdesivir, the mean duration required for reduction of illness was reduced [7.8 (+3.23) days] compared to those patients receiving the placebos plus remdesivir [8.9 (+2.95) days]. The median duration of illness was 8.0 days for the treatment group receiving the example formulation plus remdesivir, and 10.0 days for those receiving placebos plus remdesivir. The difference in these outcomes was statistically significant (p=0.030) in favor of the group of patients receiving the example formulation plus remdesivir.

The mean Severity Scores for the COVID-19-caused symptoms of nasal congestion, sore throat, cough, fatigue, headache, diarrhea, and smell/taste disorders had greater reduction in the treatment group receiving the example formulation plus remdesivir, than in the group receiving placebos plus remdesivir, and the difference was statistically significant.

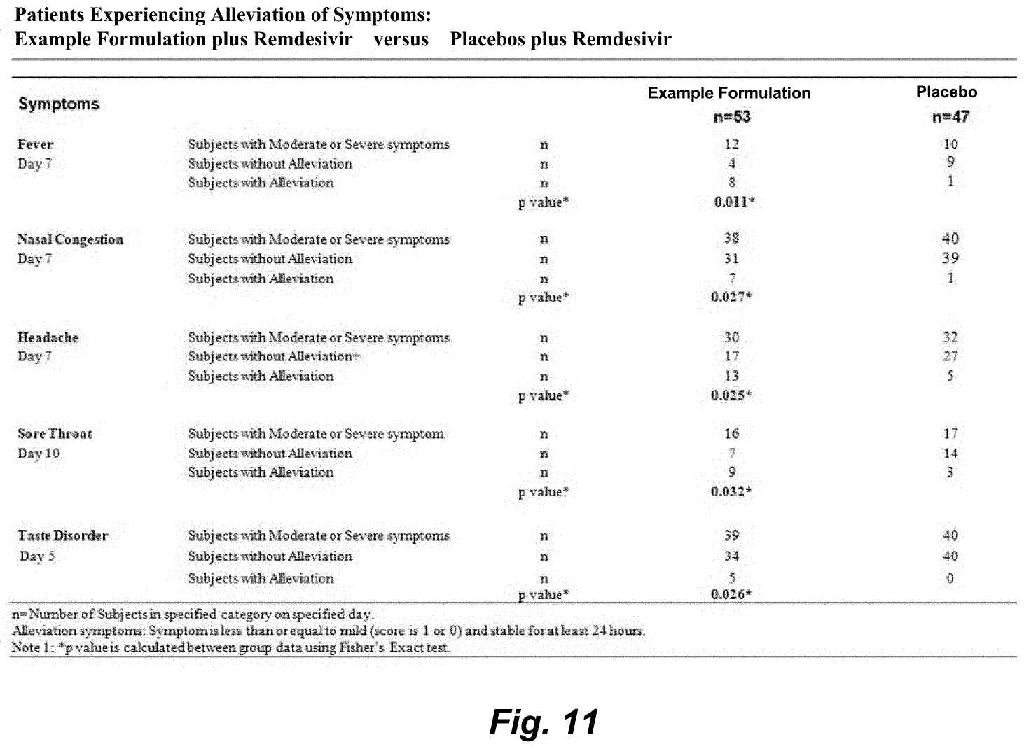

FIG. 11 shows outcomes between patients receiving the example formulation plus remdesivir, an antiviral, versus patients receiving placebos plus remdesivir. On analyzing the percentage of subjects experiencing alleviation of COVID-19 symptoms at every 24 hour interval after the first dose to the end of day 14, the results were statistically significant in favor of the example formulation plus remdesivir compared to receiving placebos plus remdesivir. Patients given the example formulation plus remdesivir had alleviation of smell and taste disorders as early as day 5 (p=0.026), alleviation of fever by day 7 (p=0.011), alleviation of nasal congestion by day 7 (p=0.027), alleviation of headache by day 7 (p=0.025), and alleviation of sore throat by day 10 (p=0.032). The mean (SD) Quality-of-Life score improved gradually in both groups by day 14 but was not statistically significant.

In subjects given the example formulation without remdesivir, the mean difference in values of the biomarker IL-6 on day 14 between the treatment group and the placebo group was statistically significant (p=0.042). The mean change in the value of the biomarker IL-6 from baseline in subjects given the example formulation without remdesivir was also statistically significant (p=0.044). On the other hand, in subjects given the example formulation with remdesivir, this same difference was not statistically significant. Incidentally, although reduction in other biomarkers were noted in the subjects given the example formulation without remdesivir, these reductions in other biomarkers were not statistically significant.

### Example Formulations

In an implementation, example extraction techniques can obtain specialized fractions from Withania somnifera and other plants. Performing custom extractions to concentrate a withaferin-A component, for example, relative to other pharmacologically active components of Withania somnifera can create preparations that target COVD-19 symptoms depending on specific physiological effect. The profile of some metabolites derived from Withania somnifera, known as withanolides, may be altered or the withanolides themselves modified by varying the soaking times, solvents, concentrations, and sequence of operations during an extraction or synthesis procedure. Withanolides are triterpene lactones forming a group of ergostane skeletal phytosteroids named after the plant. Since Withania somnifera contains many structurally diverse withanolides in its leaves as well as its roots, specific secondary metabolites may be selectively extracted and combined for physiological effect. Some of the desired withanolides are structurally distinct from tropane/nortropane alkaloids (usually found in Solanaceae plants) and are produced only by a few genera within the family Solanaceae.

A total of 62 major and minor primary and secondary metabolites from leaves, and 48 metabolites from roots have been identified in Withania somnifera. Approximately 29 of these bioactive metabolites are common to both leaf and root tissues. These metabolites also comprise fatty acids, organic acids, amino acids, sugars and sterol based compounds, for example. Some 11 bioactive sterol-lactone molecules are also available. Approximately 27 of the identified metabolites have been succinctly quantified. Highly significant qualitative and quantitative differences are noticeable between the leaf and root tissues, particularly with respect to the secondary metabolites.

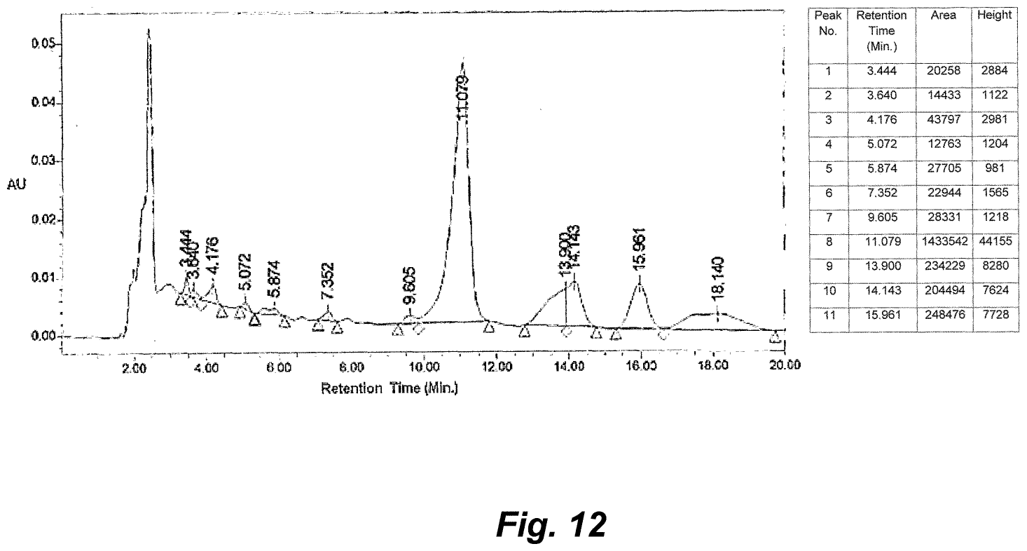

FIG. 12 shows example major and minor high-performance liquid chromatography (HPLC) peaks of select metabolites of Withania somnifera in a custom example formulation. Ten or more different peaks are evident. Out of ten different retention times, a retention time of 11.11 minutes matches withaferin-A, for example, along with other minor peaks at different minutes or retention, such as 14.20 min and 16.019 min.

An example liquid chromatography with tandem mass spectrometry (LC—MS—MS) estimation shows a good match of retention times with components such as withanolide A, 24,25,dihydroxy withanolide D, 27 deoxy withaferine A and somniferinein standards. In the LC/MS technique, the quantity of each component present can be estimated by comparing with the AUC of the respective std component. In the LCMS analysis, 10-12 different mass values are present and observable. Out of ten different mass values, the molecule depicting each mass value is usually identifiable. In an implementation, a custom example formulation includes at least a combination of withanolide-A; 24,25,dihydroxy withanolide D5-B; 6B epoxy-4Bhydroxy-1-oxo 20S; 22Rwitha 2,24dienolide i.e, 27 deoxy withaferine A and somniferinein.

Boswellia serrata provides boswellic acid, capable of inhibiting 5-lipoxygenase and leukotriene synthesis, thereby providing antiinflammatory properties among other physiological effects. As an antileukotriene and leukotriene receptor antagonist, the boswellic acid addresses (treats and prevents) dyspnea and breathing problems associated with pulmonary symptoms of COVID-19 disease.

The bioactive metabolites that can be extracted from the Zingiber officinale rhizome include [6]-gingerol (1-[4′-hydroxy-3′-methoxyphenyl]-5-hydroxy-3-decanone. Zingiber officinale can be fractioned into at least 14 bioactive compounds including [4]-gingerol, [6]-gingerol, [8]-gingerol, [10]-gingerol, [6]-paradol, [14]-shogaol, [6]-shogaol, 1-dehydro-[10]-gingerdione, [10]-gingerdione, hexahydrocurcumin, tetrahydrocurcumin, gingerenone A, 1,7-bis-(4′ hydroxyl-3′ methoxyphenyl)-5-methoxyhepthan-3-one, and methoxy-[10]-gingerol, for example. A primary metabolite, (S)-[6]-gingerol-4′ glucuronide, and other gingerols provide antioxidant and anti-inflammatory action during COVID-19 disease greatly potentiating the effects of the Withania somnifera derivatives, and also providing a pain killer.

Custom extracts of curcumin and other curcuminoids are obtainable from the rhizome of Curcuma longa. Curcumin is a polyphenol that can modulate multiple cell signaling pathways involved in multiple pathological symptoms of COVID-19 involving the cardiovascular system. Curcuma longa also contains useful alkaloids, saponins, tannins, sterols, phytic acid, flavonoids, and trace amounts of phenol, these for potentiating the mechanism of actions of other metabolites. Curcumin, other curcuminoids, and essential oils of Curcuma longa provide bioactive properties that target at least afflicted olfactory vasculature affected by COVID-19 disease.

Example Extraction and Modification of Plants and Derivatives

In an implementation, an example formulation treats long COVID sequelae. Some formulations may also alleviate cytokine storm manifestations of COVID-19 disease and reduce the viral load during the acute phase.

Example methods of extraction, for example similar to those described in U.S. Pat. No. 8,808,769 to Chitre et al., may be used to yield bioactive compounds more pharmacologically efficacious than those extracted by conventional straight extraction methods alone. Opportunistic extraction can achieve improved concentrations, purity, and modification of the ratios of bioactive species, as well as careful preservation and non-destruction of the desired active chemical species of the selected plants.

The plant material obtained from the one or more plant types may be subjected to hydro-alcoholic extraction in presence of a water-insoluble solvent, using various sequences of techniques. The hydro-alcoholic extraction may include soaking the plant material in a mixture of aqueous alcohol and a water-insoluble solvent for a predetermined time. The water-insoluble solvent used in the hydro-alcoholic extraction may be chloroform, acetone, dichloromethane, or tetra-chloromethane, for example. The mixture may be stirred occasionally while plant materials are allowed to soak. The stirring may be accomplished using methods known in the art, such as laboratory or industrial stirrer/shaker, or optionally the mixture may be stirred manually by using an appropriate stirrer. Control of pH can be used to form useful carboxylate ions from the herbal components that are carboxylic acids or phenolcarboxylic acids.

In order to extract the pharmacologically active ingredients effectively, the water-insoluble solvent and the aqueous alcohol present in the mixture should penetrate the tissues and/or cells of the plant material for a specified length of time, and with the periodic agitation or stirring.

Secondary metabolites of a given plant may then be dissolved in the solvent and further extracted. Soaking plant material in the mixture of aqueous alcohol and the water-insoluble solvent can result in extraction of the pharmacologically active ingredients of the one or more plants in two different phases. The two different phases may be a hydro-alcoholic phase and a water-insoluble phase. After predetermined soaking times are over, the mixtures can be filtered. The filtration may be carried out using the methods generally known and used in the art of liquid-liquid extraction. Alternatively, the filtration may be achieved using laboratory or industrial filtration procedures. As a result of filtering the mixture, a first residue and a first filtrate are obtained. The dry extract is subjected to one or more of a de-pigmentation process, a de-fatting process, and/or a detoxification process.

In an example formulation, the raw plant parts, such as roots of Withania somnifera, gum resin of Boswellia serrata, and rhizomes of Curcuma longa and Zingiber officinale are each gathered individually. After washing they may be minced and soaked overnight in the alcohol-water mixture, and processed further. The mixtures may be cleared up by a defatting agent before testing for standardization and before analytical testing by High Performance Liquid Chromatography (HPLC), High Pressure Thin Layer Liquid Chromatography (HPTLC) and Ultraviolet Spectrophotometry. Once four extracts, for example, are obtained further manufacture to a dosage form of tablet may also be performed as described below. Various extracts may also be further developed into liquid formulations as described further below.

### Example Tablet Manufacturing Process

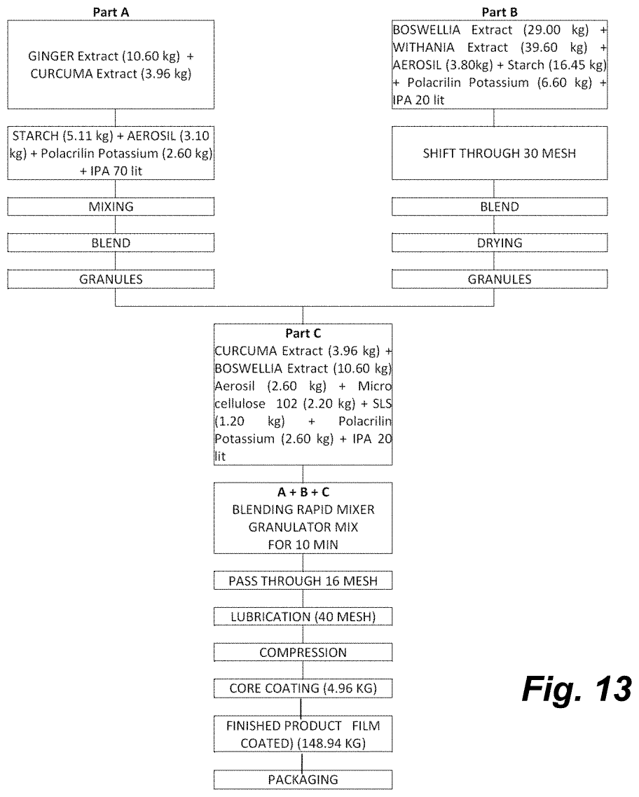

FIG. 13 shows an example process for making a batch size of approximately 220,000 tablets. Further details of the example process follow:
i. Stainless steel (SS) vessels can be prepared with 316 quality stainless steel sheets.
ii. In one process, only stainless steel scoops and spatulas are used for removing the required quantity of extracts.

### Step A. Granulation—Ginger and Curcuma

i. Weigh ginger extract (10.60 kg) and curcuma extract (3.96 kg) accurately.
ii. Pass starch maize (5.11 kg) and colloidal anhydrous silica (3.10 kg) through a 40 mesh SS sieve.
iii. Mix the above ingredients together in a suitable mass mixer for 30 min with Polacrilin potassium (2.60 kg).
iv. Add the ginger extract and curcuma extract and mix well.
v. Granulate material using isopropyl alcohol (IPA) (14 lit).
vi. Pass the above mixture thorough a 30 mesh sieve.
vii. Dry granulate at 45-50° C. until loss on drying (LOD) is 2-3% only.

### Step B. Granulation—Withania and Boswellia

i. Weigh the withania extract (39.60 kg) accurately in SS container and pass through a 16 mesh SS sieve.
ii. Weigh boswellia extract (29.00 kg) accurately in powder form in SS vessel and pass through a multi-mill 2 mm screen. Collect in a suitable SS container.
iii. Mix both extracts with colloidal silicone dioxide (e.g., Aerosil) (3.80 kg), starch maize (16.45 kg) and Polacrilin potassium (6.60 kg).
iv. Granulate above material using IPA (4.5 Lit)
v. Pass the above mix thorough a 30 mesh sieve.
vi. Dry mix for 15 min.
vii. Dry granules at 45° C.-50° C. till LOD is 2-3%

### Step C. Mixing of Granules

i. Mix Part A and Part B granules in a rapid mixer granulator for 10 min. Granulate with colloidal silicon dioxide (2.60 kg), curcuma extract (3.96 kg), boswellia extract (10.60 kg), Polacrilin potassium (2.60 kg), microcrystalline cellulose (2.20 kg) and sodium lauryl sulfate (1.20 kg) with IPA (4.5 Liters), then pass through a 40# sieve. Add in rapid mixer granulator and blend to desired consistency.
ii. Mix for 15 min.
iii. Dry the wet mass in a fluid bed dryer (FBD) at room temperature for 20 min.
iv. Pass this mass through a multi-mill using a 12 mesh sieve to form uniform granules.
v. Dry these granules formed in a fluid bed dryer with inlet air temperature of 60-70° C. until the outlet air temperature reaches 38° C. (or till the moisture content of the granules is 2-3%).
vi. Sift the semi-dried granules through a 16 mesh sieve and mill the retention through multi-mill and cad-mill using 1.5 mm screen.
vii. Load the dried granules in a double cone blender and mix for 10 mins.
viii. Sift lubrication material through a 40# sieve.
ix. Add lubrication material to dried granules and mix for 10 minutes.
x. Compress the tablets on a compression machine
xi. Complete film coating with clear colorcoat (4.96 kg) on Ganscota.

### Step D. In-Process Quality Control Procedures

In-process testing is done at the following stages:
i. Moisture content of both the granules is checked after necessary size reduction and drying. Q.A. personnel draw the composite sample.
ii. Moisture content is determined using a Karl Fischer Titrimeter, for example. Further processing is done after getting a compliance report from Q.A. (Limit for Step A granules: 2-3%, and for Step B granules: 2-3%)
iii. Moisture content of the final blend of tablets (ready for filling) is checked by Q.A. personnel, for example.
iv. During compression of tablet, the atmospheric conditions of temperature and relative humidity are monitored every hour. Average weight of the tablets is checked every 15 minutes.

In a real-world example of a basic extraction procedure having minimal steps, dried roots of Withania somnifera were obtained from a local herb supplier in Pune, India. Methanol, chloroform, and hexane were obtained from Merck, India. Water and all other reagents used were of analytical grade. The dried roots of the Withania somnifera were coarsely powdered, mechanically. The coarse powder of the matured roots of Withania somnifera thus obtained (e.g., 1 kg) was transferred to a 10 L flask. Then 3 L methanol (60% v/v) was added to the flask followed by an addition of 4 L of chloroform. The resultant mixture was allowed to soak overnight (8-12 hours). The mixture was intermittently stirred. After the 8-12 hours, the mixture was filtered to obtain the first residue and the first filtrate.

In an implementation, the first filtrate is allowed to settle. The first filtrate, once settled, has two immiscible layers. The two immiscible layers include an aqueous methanol layer and a chloroform layer. The chloroform layer is separated. Thereafter, the chloroform layer is concentrated on a rotary evaporator under reduced pressure and dried at 50° C. to obtain the chloroform soluble fraction (e.g., about 23 g). The chloroform soluble fraction thus obtained is then dissolved in hexane (2 L). The resultant solution is filtered to obtain a second residue and a second filtrate. The second residue is then dried at 50° C. and stored as a first fraction. The first residue obtained as a result of filtering the mixture is subjected to re-extraction by repeating the steps above to obtain a second fraction. The first fraction and the second fraction are mixed and stored, as an example formulation.

In another example formulation, the first fraction and second fraction as stored above are further extracted with methanol and subjected to High Performance Liquid Chromatography (HPLC) analysis.

### Example Liquid Formulation for Elderly COVID-19 patients

A liquid or syrup form of an example formulation effectively implements treatment in more vulnerable populations, such as elderly patients. For older individuals, swallowing tablets can be challenging. The liquid dosage form can be dosed with similar efficacy as the tablet form. As previously mentioned, the highest death rate from COVID-19 diseased has occurred in the elderly age group, at least in one phase of the pandemic. The option of having a liquid form with similar dosage helps the ultimate delivery and patient compliance during treatment.

To generate an example liquid form, the plants are individually extracted using a hydro glycolic extraction process. Propylene glycol is used as a co-solvent, for example as a 50% mixture in water. Propylene glycol is a colorless and odorless liquid with a sweet taste. It is used in foods, beverages and in drinks as a solubilizer, fragrance enhancer and viscosity modifier, and is widely applied in mouthwash and toothpaste. The propylene glycol offers a wide range of advantages such as, biocompatibility, biodegradability, stability, hygroscopic, non-toxic and more importantly water solubility. It also possesses bacteriostatic and fungistatic properties, and can thus act as a preservative.

The plant extracts, such as extracts of the four herbs described above, are mixed, in approximately the same ratio as in the solid tablet form described above. Concentrations of the biologically active markers are documented by High Performance Liquid Chromatography (HPLC) and by Thin Layer Chromatography (TLC). This ensures that the same activity is present in the liquid form as in the oral tablets or capsules. The mixture is a clear liquid with a pleasant taste that can be easily administered to elderly or ill patients, and for ease of use in the general population.

### Additional Physiological Effects

A high Erythrocyte Sedimentation Rate (ESR) or a high level of C-reactive protein (CRP) in the blood are markers of current inflammation. The inflammation can be caused via several pathological mechanisms effected by the SARS-CoV-2 coronavirus. Of greatest alarm, the high CRP levels can indicate inflammation in the arteries of the heart, or pulmonary inflammation, giving rise to the high mortality rate of COVID-19.

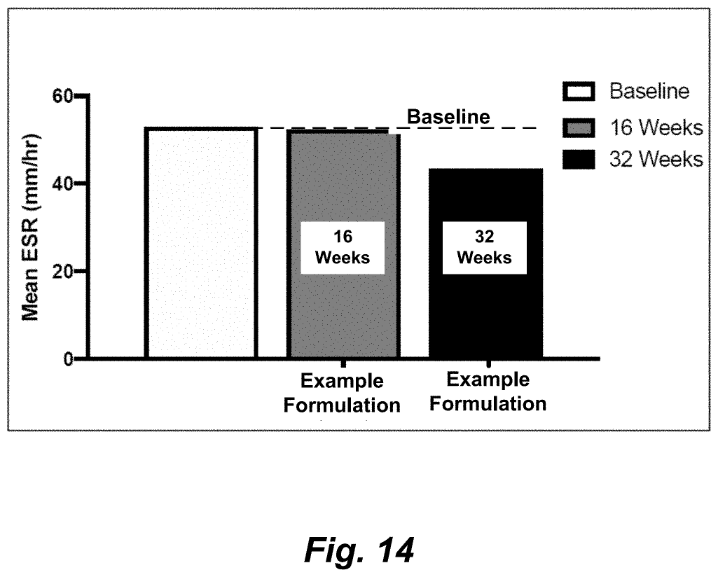

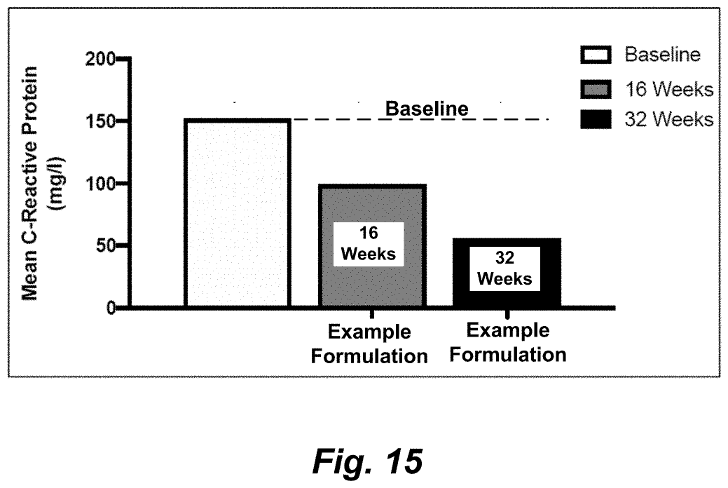

As shown in FIGS. 14-15, treatment with the example formulation for 16 weeks trended towards reduced ESR and CRP levels. This reduction is statistically significant after 32 weeks of cumulative therapy with the regimen.

The example formulation of the clinical study was found to enhance cardiorespiratory function. The level of cardiovascular health affects the rate at which oxygen can be delivered to the entire body. Readouts for cardiopulmonary and cardiorespiratory functionality include maximal cardiac output, pulmonary diffusion, blood volume, and blood flow. Measurement of maximal aerobic capacity (VO₂ max) reflects the ability of the cardiorespiratory system to transport oxygen to a long COVID patient. Significant improvement in VO₂ max was observed relative to the placebo group which demonstrated no change with respect to their baseline parameters. This feature of the example formulation helps the blood oxygen levels in long COVID patients.

The example formulation appears to block multiple proinflammatory cytokines such as IL-6 and TNF-α. The example formulation decreases the severity of long COVID sequelae and their clinical manifestation, resulting in reduced morbidity and mortality. The example formulations can also stabilize clotting factors in the body, providing prophylaxis against thromboembolic events.

As a treatment or prophylactic, the example formulations may also be combined with agents such as vitamin C, lysine, zinc, and so forth, for a safe and effective immune system boosting and stimulation of the body's defense mechanisms to aid against development of long COVID sequelae.

### Example Processes

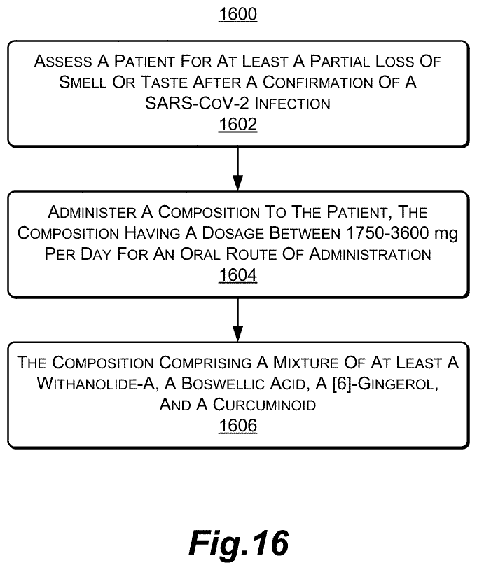

FIG. 16 shows an example method 1600 for treating at least a partial loss of smell or taste secondary to a SARS-CoV-2 infection. In FIG. 16, operations of the example method 1600 are shown in individual blocks.

At block 1602, a patient is assessed for at least a partial loss of smell or taste after a confirmation of a SARS-CoV-2 infection.

At block 1604, a composition is administered to the patient, the composition having a dosage between 1750-3600 mg per day for an oral route of administration.

At block 1606, the composition comprises a mixture of at least a withanolide-A, a boswellic acid, a [6]-gingerol, and a curcuminoid.

The example method 1600 may include administering the example composition as approximately 5.00 parts by weight of a modified derivative of a Withania somnifera herb, approximately 5.00 parts by weight of a Boswellia serrata herb, approximately 1.33 parts by weight of a Zingiber officinale rhizome, and approximately 1.00 part by weight of a Curcuma longa rhizome.

The example method 1600 may include administering the dosage of between 1750-3600 mg per day as a modified derivative of a Withania somnifera herb, an extract of a Boswellia serrata herb, an extract of a Zingiber officinale rhizome, and an extract of a Curcuma longa rhizome.

The example method 1600 may further include modifying the Withania somnifera herb to concentrate select metabolites of the Withania somnifera herb as the modified derivative.

The example method 1600 may further include modifying the Withania somnifera herb to provide an increased fraction of a withaferin-A component as the modified derivative, wherein the [6]-gingerol or the curcuminoid of the composition potentiates the withaferin-A component.

The example method 1600 may further include modifying the Withania somnifera herb to provide a non-naturally occurring mixture of bioactive components comprising withanolide-A, 24,25,dihydroxy withanolide D5-B, 6B epoxy-4Bhydroxy-1-oxo 20S, 22Rwitha 2,24dienolide, (27 deoxy withaferine A), and somniferinein as the modified derivative.

The example method 1600 may further include measuring a pro-inflammatory marker level of the patient, and titrating the dosage of the composition up or down from approximately 1750-3600 mg per day based on the pro-inflammatory marker level. For example, the pro-inflammatory marker level may be a measured interleukin-6 level.

The example method 1600 may further include co-administering an antiviral medication with the composition. For example, the antiviral medication may be nirmatrelvir or remdesivir. In an implementation, an adjuvant such as ARTOVID-20 is administered with the antiviral medication.

In the example method 1600, the full or partial loss of smell or taste may be a loss of smell associated with anosmia or a distortion of smell associated with parosmia.

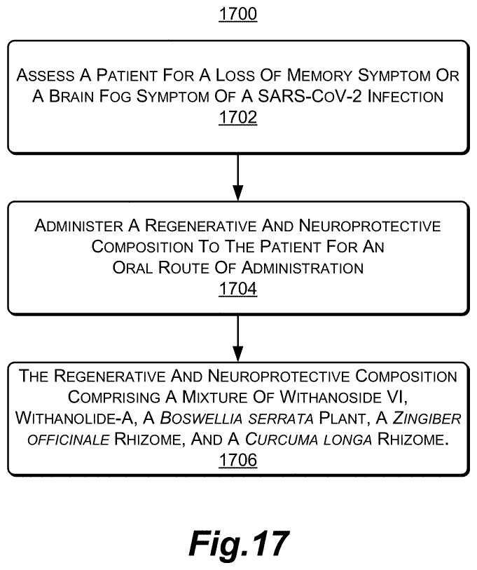

FIG. 17 shows an example method 1700 for treating a memory loss or a brain fog symptom secondary to a SARS-CoV-2 infection. In FIG. 17, operations of the example method 1700 are shown in individual blocks.

At block 1702, a patient is assessed for a loss of memory symptom or a brain fog symptom of a SARS-CoV-2 infection.

At block 1704, a regenerative and neuroprotective composition is administered to the patient via an oral route of administration.

At block 1706, the regenerative and neuroprotective composition comprises a mixture of at least withanoside VI, withanolide A, a Boswellia serrata plant, a Zingiber officinale rhizome, and a Curcuma longa rhizome.

The example method 1700 may further include administering the composition as approximately 5.00 parts by weight of at least some components of a Withania somnifera plant, approximately 5.00 parts by weight of the Boswellia serrata plant, approximately 1.33 parts by weight of the Zingiber officinale rhizome, and approximately 1.00 part by weight of the Curcuma longa rhizome.

The example method 1700 may further include dividing the dosage into 4 parts for administration 4 times per day (QID) to the patient.

The example method 1700 may further include measuring a pro-inflammatory marker level of the patient, and titrating a dosage of the composition up or down from approximately 1750-3600 mg per day based on the pro-inflammatory marker level.

The example method 1700 may further include administering the composition with an acetylcholinesterase inhibitor.

The example method 1700 may further include combining the regenerative and neuroprotective composition with one of 250 mg vitamin C, 500 mg lysine, or 10 mg zinc gluconate for oral administration to the patient.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The present invention is defined solely by the appended claims including any amendments made during the pendency of this application and all equivalents of those claims as issued.

1. A method for treating at least a partial loss of smell or taste secondary to a SARS-CoV-2 infection, comprising:
assessing a patient for at least a partial loss of smell or taste after a confirmation of a SARS-CoV-2 infection;
administering a composition to the patient, the composition having a dosage between 1750-3600 mg per day for an oral route of administration;
wherein the composition comprises a mixture of at least a withanolide-A, a boswellic acid, a [6]-gingerol, and a curcuminoid.

2. The method of claim 1, wherein administering the composition comprises administering approximately 5.00 parts by weight of a modified derivative of a Withania somnifera herb, approximately 5.00 parts by weight of a Boswellia serrata herb, approximately 1.33 parts by weight of a Zingiber officinale rhizome, and approximately 1.00 part by weight of a Curcuma longa rhizome.

3. The method of claim 1, wherein administering the dosage between 1750-3600 mg per day of a composition to the patient comprises administering a modified derivative of a Withania somnifera herb, an extract of a Boswellia serrata herb, an extract of a Zingiber officinale rhizome, and an extract of a Curcuma longa rhizome.

4. The method of claim 3, further comprising modifying the Withania somnifera herb to concentrate select metabolites of the Withania somnifera herb as the modified derivative.

5. The method of claim 3, further comprising modifying the Withania somnifera herb to provide an increased fraction of a withaferin-A component as the modified derivative, wherein the [6]-gingerol or the curcuminoid potentiates the withaferin-A component.

6. The method of claim 3, further comprising modifying the Withania somnifera herb to provide a non-naturally occurring mixture of bioactive components comprising withanolide-A, 24,25,dihydroxy withanolide D5-B, 6B epoxy-4Bhydroxy-1-oxo 20S, 22Rwitha 2,24dienolide, (27 deoxy withaferine A), and somniferinein as the modified derivative.

7. The method of claim 1, further comprising measuring a pro-inflammatory marker level of the patient; and
titrating the dosage of the composition up or down from approximately 1750-3600 mg per day based on the pro-inflammatory marker level.

8. The method of claim 7, wherein the pro-inflammatory marker level comprises an interleukin-6 level.

9. The method of claim 1, further comprising co-administering an antiviral medication with the composition.

10. The method of claim 9, wherein the antiviral medication comprises nirmatrelvir or remdesivir.

11. The method of claim 1, wherein the at least partial loss of smell or taste comprises a loss of smell associated with anosmia or a distortion of smell associated with parosmia.

12. A method for treating at least a partial loss of smell or taste secondary to a SARS-CoV-2 infection, comprising:
assessing a patient for the at least partial loss of smell or taste after a confirmation of a SARS-CoV-2 infection;
administering a prescription antiviral agent to the patient; and
administering ARTOVID-20 tablets to the patient as an adjuvant agent with the prescription antiviral agent.

13. The method of claim 12, wherein the antiviral agent comprises nirmatrelvir or remdesivir.

14. The method of claim 12, further comprising:
measuring a pro-inflammatory marker level of the patient; and
titrating a dosage of the ARTOVID-20 tablets based on the pro-inflammatory marker level.

15. A method for treating a memory loss or a brain fog symptom secondary to a SARS-CoV-2 infection, comprising:
assessing a patient for a loss of memory symptom or a brain fog symptom of a SARS-CoV-2 infection;
administering a regenerative and neuroprotective composition to the patient for an oral route of administration; and
wherein the composition comprises a mixture of withanoside VI, withanolide A, a Boswellia serrata plant, a Zingiber officinale rhizome, and a Curcuma longa rhizome.

16. The method of claim 15, further comprising administering a dosage between 1750-3600 mg per day of the composition, the composition comprising approximately 5.00 parts by weight of at least some components of a Withania somnifera plant, approximately 5.00 parts by weight of the Boswellia serrata plant, approximately 1.33 parts by weight of the Zingiber officinale rhizome, and approximately 1.00 part by weight of the Curcuma longa rhizome.

17. The method of claim 16, further comprising dividing the dosage into 4 parts for administration 4 times per day (QID) to the patient.

18. The method of claim 16, further comprising:
measuring a pro-inflammatory marker level of the patient; and
titrating a dosage of the composition up or down from approximately 1750-3600 mg per day based on the pro-inflammatory marker level.

19. The method of claim 15, further comprising administering the composition with an acetylcholinesterase inhibitor.

20. The method of claim 15, further comprising combining the composition with one of 250 mg vitamin C, 500 mg lysine, or 10 mg zinc gluconate for oral administration to the patient.
